SEQUENCE LISTING (70 sequences)

SEQ ID NO: 1 - DNA, 33 bp, Homo sapiens
  source 1..33: mol_type = other DNA
tcggtcagaggctagctacaacgatgcgttgct

SEQ ID NO: 2 - DNA, 33 bp, Homo sapiens
  source 1..33: mol_type = other DNA
ggcgtacgaggctagctacaacgactgctcggt

SEQ ID NO: 3 - DNA, 33 bp, Homo sapiens
  source 1..33: mol_type = other DNA
ggcggcgtaggctagctacaacgagacctgctc

SEQ ID NO: 4 - DNA, 33 bp, Homo sapiens
  source 1..33: mol_type = other DNA
ctcgggtcaggctagctacaacgactgggtagc

SEQ ID NO: 5 - DNA, 33 bp, Homo sapiens
  source 1..33: mol_type = other DNA
tcctctgcaggctagctacaacgacggggtcct

SEQ ID NO: 6 - DNA, 33 bp, Homo sapiens
  source 1..33: mol_type = other DNA
actctgcaaggctagctacaacgatctgcgagc

SEQ ID NO: 7 - DNA, 33 bp, Homo sapiens
  source 1..33: mol_type = other DNA
gggcgacgaggctagctacaacgatctgcaatt

SEQ ID NO: 8 - DNA, 33 bp, Homo sapiens
  source 1..33: mol_type = other DNA
aaggggcgaggctagctacaacgagactctgca

SEQ ID NO: 9 - DNA, 33 bp, Homo sapiens
  source 1..33: mol_type = other DNA
aaaacgggaggctagctacaacgacaggttgta

SEQ ID NO: 10 - DNA, 33 bp, Homo sapiens
  source 1..33: mol_type = other DNA
agaataaaaggctagctacaacgagggaccagg

SEQ ID NO: 11 - DNA, 33 bp, Homo sapiens
  source 1..33: mol_type = other DNA
atggcagaaggctagctacaacgaaaaacggga

SEQ ID NO: 12 - DNA, 33 bp, Homo sapiens
  source 1..33: mol_type = other DNA
aactgggtaggctagctacaacgaggcagaata

SEQ ID NO: 13 - DNA, 33 bp, Homo sapiens
  source 1..33: mol_type = other DNA
atccaaaaaggctagctacaacgatgggtatgg

SEQ ID NO: 14 - DNA, 33 bp, Homo sapiens
  source 1..33: mol_type = other DNA
aggggaagaggctagctacaacgaaaaaatcca

SEQ ID NO: 15 - DNA, 33 bp, Homo sapiens
  source 1..33: mol_type = other DNA
ttttaaaaaggctagctacaacgatatcttgga

SEQ ID NO: 16 - DNA, 33 bp, Homo sapiens
  source 1..33: mol_type = other DNA
gtggggggaggctagctacaacgagggaaggct

SEQ ID NO: 17 - DNA, 33 bp, Homo sapiens
  source 1..33: mol_type = other DNA
gttgaatgaggctagctacaacgattgctttcg

SEQ ID NO: 18 - DNA, 33 bp, Homo sapiens
  source 1..33: mol_type = other DNA
gtcgttgaaggctagctacaacgagatttgctt

SEQ ID NO: 19 - DNA, 33 bp, Homo sapiens
  source 1..33: mol_type = other DNA
ggcccggaaggctagctacaacgaccgcgcgcg

SEQ ID NO: 20 - DNA, 33 bp, Homo sapiens
  source 1..33: mol_type = other DNA
tcacctccaggctagctacaacgaggcctcggc

SEQ ID NO: 21 - DNA, 33 bp, Homo sapiens
  source 1..33: mol_type = other DNA
ccgccgtcaggctagctacaacgactccatggc

SEQ ID NO: 22 - DNA, 33 bp, Homo sapiens
  source 1..33: mol_type = other DNA
ggtggctcaggctagctacaacgaccagcgcgg

SEQ ID NO: 23 - DNA, 33 bp, Homo sapiens
  source 1..33: mol_type = other DNA
cgttgagcaggctagctacaacgaggcggggtg

SEQ ID NO: 24 - DNA, 33 bp, Homo sapiens
  source 1..33: mol_type = other DNA
ccgcgtccaggctagctacaacgagtaggagtg

SEQ ID NO: 25 - DNA, 33 bp, Homo sapiens
  source 1..33: mol_type = other DNA
cagcgggtaggctagctacaacgatgcgccgcg

SEQ ID NO: 26 - DNA, 33 bp, Homo sapiens
  source 1..33: mol_type = other DNA
gcacatccaggctagctacaacgactcctccgg

SEQ ID NO: 27 - DNA, 33 bp, Homo sapiens
  source 1..33: mol_type = other DNA
aaaagcacaggctagctacaacgaccacctcct

SEQ ID NO: 28 - DNA, 33 bp, Homo sapiens
  source 1..33: mol_type = other DNA
taaaaagcaggctagctacaacgaatccacctc

SEQ ID NO: 29 - DNA, 33 bp, Homo sapiens
  source 1..33: mol_type = other DNA
gaccgtcgaggctagctacaacgagttaaaaag

SEQ ID NO: 30 - DNA, 33 bp, Homo sapiens
  source 1..33: mol_type = other DNA
ttgccttgaggctagctacaacgacgtcgatgt

SEQ ID NO: 31 - DNA, 33 bp, Homo sapiens
  source 1..33: mol_type = other DNA
agggcgggaggctagctacaacgagtggttgcc

SEQ ID NO: 32 - DNA, 33 bp, Homo sapiens
  source 1..33: mol_type = other DNA
tggccctgaggctagctacaacgacgagtttcc

SEQ ID NO: 33 - DNA, 33 bp, Homo sapiens
  source 1..33: mol_type = other DNA
acctctgcaggctagctacaacgacgtggccct

SEQ ID NO: 34 - DNA, 33 bp, Homo sapiens
  source 1..33: mol_type = other DNA
cggagggtaggctagctacaacgactctgcacc

SEQ ID NO: 35 - DNA, 33 bp, Homo sapiens
  source 1..33: mol_type = other DNA
ggcggcacaggctagctacaacgactggctccc

SEQ ID NO: 36 - DNA, 33 bp, Homo sapiens
  source 1..33: mol_type = other DNA
cgggcggcaggctagctacaacgaacctggctc

SEQ ID NO: 37 - DNA, 33 bp, Homo sapiens
  source 1..33: mol_type = other DNA
agggatccaggctagctacaacgagaagcagag

SEQ ID NO: 38 - DNA, 33 bp, Homo sapiens
  source 1..33: mol_type = other DNA
gggtagggaggctagctacaacgaccatgaagc

SEQ ID NO: 39 - DNA, 33 bp, Homo sapiens
  source 1..33: mol_type = other DNA
gggctgagaggctagctacaacgatccaggggg

SEQ ID NO: 40 - DNA, 33 bp, Homo sapiens
  source 1..33: mol_type = other DNA
gtggatggaggctagctacaacgagtcttggag

SEQ ID NO: 41 - DNA, 33 bp, Homo sapiens
  source 1..33: mol_type = other DNA
cgtggtggaggctagctacaacgaggacgtctt

SEQ ID NO: 42 - DNA, 33 bp, Homo sapiens
  source 1..33: mol_type = other DNA
gggggtagaggctagctacaacgaggagagggg

SEQ ID NO: 43 - DNA, 33 bp, Homo sapiens
  source 1..33: mol_type = other DNA
ggaggaggaggctagctacaacgagaggccggg

SEQ ID NO: 44 - DNA, 33 bp, Homo sapiens
  source 1..33: mol_type = other DNA
gccccccgaggctagctacaacgaaaggaggag

SEQ ID NO: 45 - DNA, 33 bp, Homo sapiens
  source 1..33: mol_type = other DNA
ccggggagaggctagctacaacgagtccttcgg

SEQ ID NO: 46 - DNA, 33 bp, Homo sapiens
  source 1..33: mol_type = other DNA
ggacagcgaggctagctacaacgagggtccggg

SEQ ID NO: 47 - DNA, 33 bp, Homo sapiens
  source 1..33: mol_type = other DNA
tggggtggaggctagctacaacgaagcgatggg

SEQ ID NO: 48 - DNA, 33 bp, Homo sapiens
  source 1..33: mol_type = other DNA
cttgaggcaggctagctacaacgatctttctcg

SEQ ID NO: 49 - DNA, 33 bp, Homo sapiens
  source 1..33: mol_type = other DNA
cacctggtaggctagctacaacgattgaggcac

SEQ ID NO: 50 - DNA, 33 bp, Homo sapiens
  source 1..33: mol_type = other DNA
gcaggggcaggctagctacaacgactggtactt

SEQ ID NO: 51 - DNA, 33 bp, Homo sapiens
  source 1..33: mol_type = other DNA
ccagcttcaggctagctacaacgagctgtcggg

SEQ ID NO: 52 - DNA, 33 bp, Homo sapiens
  source 1..33: mol_type = other DNA
gtgggacgaggctagctacaacgatccagcttc

SEQ ID NO: 53 - DNA, 33 bp, Homo sapiens
  source 1..33: mol_type = other DNA
ggagtgggaggctagctacaacgagactccagc

SEQ ID NO: 54 - DNA, 33 bp, Homo sapiens
  source 1..33: mol_type = other DNA
atgctgccaggctagctacaacgagggagtggg

SEQ ID NO: 55 - DNA, 33 bp, Homo sapiens
  source 1..33: mol_type = other DNA
gggcggtcaggctagctacaacgagctgccacg

SEQ ID NO: 56 - DNA, 33 bp, Homo sapiens
  source 1..33: mol_type = other DNA
gaggctccaggctagctacaacgaccagggcgg

SEQ ID NO: 57 - DNA, 33 bp, Homo sapiens
  source 1..33: mol_type = other DNA
gtgggtcgaggctagctacaacgagaggaggct

SEQ ID NO: 58 - DNA, 33 bp, Homo sapiens
  source 1..33: mol_type = other DNA
aggtggtgaggctagctacaacgaggggtggtg

SEQ ID NO: 59 - DNA, 33 bp, Homo sapiens
  source 1..33: mol_type = other DNA
actcgggcaggctagctacaacgagtagggcgg

SEQ ID NO: 60 - DNA, 33 bp, Homo sapiens
  source 1..33: mol_type = other DNA
ggagctgtaggctagctacaacgatcgggcacg

SEQ ID NO: 61 - DNA, 33 bp, Homo sapiens
  source 1..33: mol_type = other DNA
ggacttgcaggctagctacaacgaccgaagccg

SEQ ID NO: 62 - DNA, 33 bp, Homo sapiens
  source 1..33: mol_type = other DNA
gggcctggaggctagctacaacgattgcatccg

SEQ ID NO: 63 - DNA, 33 bp, Homo sapiens
  source 1..33: mol_type = other DNA
tgtgctggaggctagctacaacgacgggccttg

SEQ ID NO: 64 - DNA, 33 bp, Homo sapiens
  source 1..33: mol_type = other DNA
gttcacacaggctagctacaacgatccctgcct

SEQ ID NO: 65 - DNA, 33 bp, Homo sapiens
  source 1..33: mol_type = other DNA
cagttcacaggctagctacaacgaactccctgc

SEQ ID NO: 66 - DNA, 33 bp, Homo sapiens
  source 1..33: mol_type = other DNA
cacagttcaggctagctacaacgaacactccct

SEQ ID NO: 67 - DNA, 33 bp, Homo sapiens
  source 1..33: mol_type = other DNA
gttgccccaggctagctacaacgaagttcacac

SEQ ID NO: 68 - DNA, 33 bp, Homo sapiens
  source 1..33: mol_type = other DNA
tcgccgccaggctagctacaacgaagtggggtc

SEQ ID NO: 69 - DNA, 33 bp, Homo sapiens
  source 1..33: mol_type = other DNA
cccgtgccaggctagctacaacgactcgccgcc

SEQ ID NO: 70 - DNA, 33 bp, Homo sapiens
  source 1..33: mol_type = other DNA
ggcgttgcaggctagctacaacgaaggtagtgt

(19) United States
(12) Patent Application Publication
(10) Pub. No.: US 2024/0382430 A1
(43) Pub. Date: Nov. 21, 2024

(54) DNAZYME HYDROGEL FORMULATIONS

(71) Applicant: Sterna Biologicals GmbH, Marburg (DE)

(72) Inventors: Thomas Rupp, Marburg (DE); Daniel Bock, Marburg (DE); Rolf Krattenmacher, Marburg (DE); Georgios Sarigiannis, Marburg (DE); Caria Prata, Marburg (DE)

(21) Appl. No.: 18/693,537

(22) PCT Filed: Sep. 28, 2022
(86) PCT No.: PCT/EP2022/076980

§ 371 Date: Mar. 20, 2024

(30) Foreign Application Priority Data
Sep. 30, 2021 (EP) 21200083.0

(51) Int. Cl. A61K 9/50; A61K 9/02; C12N 15/113

(52) U.S. Cl. CPC A61K 9/5031; A61K 9/02; C12N 15/113; C12N 2310/127

(57) ABSTRACT

The invention provides a pharmaceutical composition comprising: a) a high molecular weight poloxamer; b) a low molecular weight poloxamer; c) a DNAzym; d) a pharmaceutically acceptable buffer; wherein the composition is liquid at room temperature or below and solid or semisolid at a temperature of 36° C. or higher. The composition is particular suitable for the treatment of intestinal diseases, in particular inflammatory intestinal diseases.

### BACKGROUND OF THE INVENTION

A DNAzyme is a catalytically active, single-stranded, synthetic DNA molecule, which does not occur in nature. Synthetic, catalytically active DNA molecules are designed as novel therapeutic moieties to specifically bind and cleave disease-related mRNA in a catalytic manner similar to enzymes (Garn & Renz, Eur. J. Immunology, 47, (2017) 22-30). These moieties therefore are DNA molecules with enzymatic activities, therefore named DNAzymes.

DNAzymes of the 10-23 family which represent a new class of anti-sense molecules were developed in the 1990s. The term “10-23 family” refers to a general DNAzyme model (Sontoro & Joyce, Proc. Natl. Acad. Sci. U.S.A., 94 (1997) 4262-4266). DNAzymes of the 10-23 model—also referred to as “10-23 DNAzymes” have a catalytic domain of 15 deoxyribonucleotides, which are flanked by two substrate binding domains (e.g., WO 2005/033314).

Potential advantages of DNAzymes include a relatively high stability against chemical or enzymatic degradation when bound to the target mRNA, a high target specificity and no dependance on intracellular enzymes.

Problems that can arise when using unmodified DNAzymes as active substances are often due to the sensitivity of the single-stranded nucleic acids against enzymatic degradation when not bound to the target mRNA and unsuitably formulated. Unless DNAzymes are present in physiologically favorable formulations, nucleic acids tend to degrade rapidly, for example through enzymatic degradation or physical stress.

Furthermore, DNAzymes can differ in their specificity and thus in their effectiveness as active agents. In addition, the stability of DNAzymes can be affected, especially by changes in the pH of the DNAzyme-containing solutions. For example, a low pH (acidic environment) can result in depurination of the DNAzyme molecules. A high pH value (basic environment) can also influence the stability and, thus, the functionality of the DNAzymes, for example by changing secondary structures of the molecules.

The preparation of suitable pharmaceutical compositions comprising DNAzymes is challenging. For example, compositions comprising the DNAzyme hgd40, a DNAzyme specifically designed to inhibit the expression of the GATA-3 transcription factor by catalytic cleavage of the GATA-3 mRNA, show an exponential increase in viscosity with higher concentrations of the DNAzyme, rendering some compositions unsuitable for specific applications; see for example EP3501607A1. As such, it is difficult to provide compositions comprising higher DNAzyme concentrations in other than aqueous compositions.

DNAzymes, like hgd40, can be used in the treatment of chronic inflammatory intestinal diseases; see EP3514235A1. In this case the DNAzyme is administered rectally in the form of an enema of an aqueous solution. Administration of drugs as liquid enema is a usual method for the treatment of inflammatory intestinal diseases. However, liquid enemas have limitations such as inconvenient administration, drug leakage and poor patient compliance.

Therefore, the object of the invention was to provide a new formulation for DNAzymes, which allow a mitigation of the issues while allowing for a suitable DNAzyme concentration.

### SUMMARY OF THE INVENTION

In a first aspect, the invention relates to invention relates to a pharmaceutical composition comprising:
a) a high molecular weight poloxamer;
b) a low molecular weight poloxamer;
c) a DNAzym;
d) a pharmaceutically acceptable buffer;

wherein the composition is liquid at room temperature or below and solid or semisolid at a temperature of 36° C. or higher.

In a further aspect, the present invention relates to a composition as defined above for use in the treatment of an intestinal disease, in particular an inflammatory intestinal disease.

### DESCRIPTION OF THE DRAWINGS

FIG. 1 shows the absorbance spectra of hgd40 released from a hydrogel composition at different time points.

FIG. 2A shows a AEX chromatography profile of a hgd40 stock solution. FIG. 2B shows an AEX chromatography profile of a hgd40 solution after hydrogel release.

### DETAILED DESCRIPTION OF THE INVENTION

The inventors of the present invention found that it is possible to formulate DNAzymes in hydrogel compositions with low gelation times, sufficiently high concentrations of the active pharmaceutical ingredient (API) and a properly adjusted T_{sol-gel} temperature. Furthermore the formed gel is additionally maintaining its integrity for a desired period of time. These formulations are therefore advantageous to liquid enema formulations. Therefore, the present invention relates in a first aspect to a pharmaceutical composition comprising a DNAzyme suitable for rectal administration. The composition is a hydrogel composition which is liquid at room temperature and below and is a solid or semisolid gel at about 36° C. or higher.

In the context of the present invention, the term room temperature refers to a temperature of 16 to 25° C., in particular 16 to 21° C. In some embodiments room temperature refers to a temperature of 21° C.

The inventors found that poloxamer-based hydrogels can be used to formulate DNAzymes in a hydrogel composition which is liquid at room temperature and below and a solid or semisolid gel at 36° and above.

Therefore, in a first aspect, the invention relates to a pharmaceutical composition comprising:
a) a high molecular weight poloxamer;
b) a low molecular weight poloxamer;
c) a DNAzym;
d) a pharmaceutically acceptable buffer;
   wherein the composition is liquid at room temperature and solid or semisolid at a temperature of 36° C. or higher.

The composition may comprise other pharmaceutically acceptable compounds, in particular excipients and/or stabilizers.

The term poloxamer refers to a class of nonionic triblock copolymers composed of a central hydrophobic chain of polyoxypropylene (poly (propylene oxide)) flanked by two hydrophilic chains of polyoxyethylene (poly (ethylene oxide)). The general structure of a poloxamer is as follows:

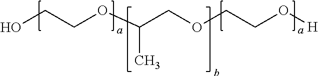

The term high molecular weight poloxamer refers to a poloxamer with a polyoxypropylene mass of 3000 g/mol or higher. The term low molecular weight poloxamer refers a poloxamer with a polyoxypropylene mass of less than 3000 g/mol.

A preferred high molecular weight poloxamer is poloxamer 407, sometimes also referred to under the tradename Pluronic F127 and a preferred low-molecular weight poloxamer is poloxamer 188, sometimes referred to under the tradename Pluronic F68.

In general the three digits in the name of the Poloxamer are used to define said poloxamer. The first two digits multiplied by 100 give the approximate molecular mass of the polyoxypropylene core, and the last digit multiplied by 10 gives the percentage polyoxyethylene content. Poloxamer 407 refers to a poloxamer with a polyoxypropylene molecular mass of 4000 g/mol and 70% polyoxyethylene content. Poloxamer 188 refers to a poloxamer with a polyoxypropylene molecular mass of 1800 g/mol and 80% polyoxyethylene content.

Accordingly, in a preferred embodiment, the invention relates to a pharmaceutical composition comprising:
a) poloxamer 407;
b) poloxamer 188;
c) a DNAzym;
d) a pharmaceutically acceptable buffer;
   wherein the composition is liquid at room temperature and solid or semisolid at a temperature of 36° C. or higher.

The inventors found that it is particularly difficult to provide a gel-forming composition which comprises a high concentration of DNAzyme. The inventors found that in particular DNAzymes that specifically inhibit the expression of the GATA-3 transcription factor are difficult to formulate due to the unique properties of these DNAzymes.

As such, in a preferred embodiment, the composition comprises a DNAzyme that specifically inhibits GATA-3 expression. In some embodiments the DNAzym is selected from the group comprising:

hgd1
SEQ ID No: 1
5′-TCGGTCAGAggctagctacaacgaTGCGTTGCT-3′
hgd2
SEQ ID No: 2
5′-GGCGTACGAggctagctacaacgaCTGCTCGGT-3′
hgd3
SEQ ID No: 3
5′-GGCGGCGTAggctagctacaacgaGACCTGCTC-3′
hgd4
SEQ ID No: 4
5′-CTCGGGTCAggctagctacaacgaCTGGGTAGC-3′
hgd5
SEQ ID No: 5
5′-TCCTCTGCAggctagctacaacgaCGGGGTCCT-3′
hgd6
SEQ ID No: 6
5′-ACTCTGCAAggctagctacaacgaTCTGCGAGC-3′
hgd7
SEQ ID No: 7
5′-GGGCGACGAggctagctacaacgaTCTGCAATT-3′
hgd8
SEQ ID No: 8
5′-AAGGGGCGAggctagctacaacgaGACTCTGCA-3′
hgd9
SEQ ID No: 9
5′-AAAACGGGAggctagctacaacgaCAGGTTGTA-3′
hgd10
SEQ ID No: 10
5′-AGAATAAAAggctagctacaacgaGGGACCAGG-3′
hgd11
SEQ ID No: 11
5′-ATGGCAGAAggctagctacaacgaAAAACGGGA-3′
hgd12
SEQ ID No: 12
5′-AACTGGGTAggctagctacaacgaGGCAGAATA-3′
hgd13
SEQ ID No: 13
5′-ATCCAAAAAggctagctacaacgaTGGGTATGG-3′
hgd14
SEQ ID No: 14
5′-AGGGGAAGAggctagctacaacgaAAAAATCCA-3′
hgd15
SEQ ID No: 15
5′-TTTTAAAAAggctagctacaacgaTATCTTGGA-3′
hgd16
SEQ ID No: 16
5′-GTGGGGGGAggctagctacaacgaGGGAAGGCT-3′
hgd17
SEQ ID No: 17
5′-GTTGAATGAggctagctacaacgaTTGCTTTCG-3′
hgd18
SEQ ID No: 18
5′-GTCGTTGAAggctagctacaacgaGATTTGCTT-3′
hgd19
SEQ ID No: 19
5′-GGCCCGGAAggctagctacaacgaCCGCGCGCG-3′
hgd20
SEQ ID No: 20
5′-TCACCTCCAggctagctacaacgaGGCCTCGGC-3′
hgd21
SEQ ID No: 21
5′-CCGCCGTCAggctagctacaacgaCTCCATGGC-3′
hgd22
SEQ ID No: 22
5′-GGTGGCTCAggctagctacaacgaCCAGCGCGG-3′
hgd23
SEQ ID No: 23
5′-CGTTGAGCAggctagctacaacgaGGCGGGGTG-3′
hgd24
SEQ ID No: 24
5′-CCGCGTCCAggctagctacaacgaGTAGGAGTG-3′
hgd25
SEQ ID No: 25
5′-CAGCGGGTAggctagctacaacgaTGCGCCGCG-3′
hgd26
SEQ ID No: 26
5′-GCACATCCAggctagctacaacgaCTCCTCCGG-3′
hgd27
SEQ ID No: 27
5′-AAAAGCACAggctagctacaacgaCCACCTCCT-3′
hgd28
SEQ ID No: 28
5′-TAAAAAGCAggctagctacaacgaATCCACCTC-3′
hgd29
SEQ ID No: 29
5′-GACCGTCGAggctagctacaacgaGTTAAAAAG-3′
hgd30
SEQ ID No: 30
5′-TTGCCTTGAggctagctacaacgaCGTCGATGT-3′
hgd31
SEQ ID No: 31
5′-AGGGCGGGAggctagctacaacgaGTGGTTGCC-3′
hgd32
SEQ ID No: 32
5′-TGGCCCTGAggctagctacaacgaCGAGTTTCC-3′
hgd33
SEQ ID No: 33
5′-ACCTCTGCAggctagctacaacgaCGTGGCCCT-3′
hgd34
SEQ ID No: 34
5′-CGGAGGGTAggctagctacaacgaCTCTGCACC-3′
hgd35
SEQ ID No: 35
5′-GGCGGCACAggctagctacaacgaCTGGCTCCC-3′
hgd36
SEQ ID No: 36
5′-CGGGCGGCAggctagctacaacgaACCTGGCTC-3′
hgd37
SEQ ID No: 37
5′-AGGGATCCAggctagctacaacgaGAAGCAGAG-3′
hgd38
SEQ ID No: 38
5′-GGGTAGGGAggctagctacaacgaCCATGAAGC-3′
hgd39
SEQ ID No: 39
5′-GGGCTGAGAggctagctacaacgaTCCAGGGGG-3′
hgd40
SEQ ID No: 40
5′-GTGGATGGAggctagctacaacgaGTCTTGGAG-3′
hgd41
SEQ ID No: 41
5′-CGTGGTGGAggctagctacaacgaGGACGTCTT-3′
hgd42
SEQ ID No: 42
5′-GGGGGTAGAggctagctacaacgaGGAGAGGGG-3′
hgd43
SEQ ID No: 43
5′-GGAGGAGGAggctagctacaacgaGAGGCCGGG-3′
hgd44
SEQ ID No: 44
5′-GCCCCCCGAggctagctacaacgaAAGGAGGAG-3′
hgd45
SEQ ID No: 45
5′-CCGGGGAGAggctagctacaacgaGTCCTTCGG-3′
hgd46
SEQ ID No: 46
5′-GGACAGCGAggctagctacaacgaGGGTCCGGG-3′
hgd47
SEQ ID No: 47
5′-TGGGGTGGAggctagctacaacgaAGCGATGGG-3′
hgd48
SEQ ID No: 48
5′-CTTGAGGCAggctagctacaacgaTCTTTCTCG-3′
hgd49
SEQ ID No: 49
5′-CACCTGGTAggctagctacaacgaTTGAGGCAC-3′
hgd50
SEQ ID No: 50
5′-GCAGGGGCAggctagctacaacgaCTGGTACTT-3′
hgd51
SEQ ID No: 51
5′-CCAGCTTCAggctagctacaacgaGCTGTCGGG-3′
hgd52
SEQ ID No: 52
5′-GTGGGACGAggctagctacaacgaTCCAGCTTC-3′
hgd53
SEQ ID No: 53
5′-GGAGTGGGAggctagctacaacgaGACTCCAGC-3′
hgd54
SEQ ID No: 54
5′-ATGCTGCCAggctagctacaacgaGGGAGTGGG-3′
hgd55
SEQ ID No: 55
5′-GGGCGGTCAggctagctacaacgaGCTGCCACG-3′
hgd56
SEQ ID No: 56
5′-GAGGCTCCAggctagctacaacgaCCAGGGCGG-3′
hgd57
SEQ ID No: 57
5′-GTGGGTCGAggctagctacaacgaGAGGAGGCT-3′
hgd58
SEQ ID No: 58
5′-AGGTGGTGAggctagctacaacgaGGGGTGGTG-3′
hgd59
SEQ ID No: 59
5′-ACTCGGGCAggctagctacaacgaGTAGGGCGG-3′
hgd60
SEQ ID No: 60
5′-GGAGCTGTAggctagctacaacgaTCGGGCACG-3′
hgd61
SEQ ID No: 61
5′-GGACTTGCAggctagctacaacgaCCGAAGCCG-3′
hgd62
SEQ ID No: 62
5′-GGGCCTGGAggctagctacaacgaTTGCATCCG-3′
hgd63
SEQ ID No: 63
5′-TGTGCTGGAggctagctacaacgaCGGGCCTTG-3′
hgd64
SEQ ID No: 64
5′-GTTCACACAggctagctacaacgaTCCCTGCCT-3′
hgd65
SEQ ID No: 65
5′-CAGTTCACAggctagctacaacgaACTCCCTGC-3′
hgd66
SEQ ID No: 66
5′-CACAGTTCAggctagctacaacgaACACTCCCT-3′
hgd67
SEQ ID No: 67
5′-GTTGCCCCAggctagctacaacgaAGTTCACAC-3′
hgd68
SEQ ID No: 68
5′-TCGCCGCCAggctagctacaacgaAGTGGGGTC-3′
hgd69
SEQ ID No: 69
5′-CCCGTGCCAggctagctacaacgaCTCGCCGCC-3′
hgd70
SEQ ID No: 70
5′-GGCGTTGCAggctagctacaacgaAGGTAGTGT-3′

In particular embodiments, the composition comprises the DNAzyme hgd40 (SEQ ID NO 40).

The present invention allows for hydrogel formulations comprising DNAzymes at higher concentrations that are suitable for medical applications. As such, it is preferred that the DNAzym concentration in the composition is at least 4 mg/mL. Therefore, in some embodiments, the DNAzyme concentration is at least 4 mg/mL. In some embodiments the DNAzym concentration is in the range from 4 mg/mL to 20 mg/mL.

In some embodiments of the invention, the DNAzym concentration is at least 8 mg/mL. In some embodiments the DNAzym concentration is in the range of 8 and 20 mg/mL, preferably in the range to 8 to 16 mg/mL.

In some embodiments, the DNAzym concentration is 4, 8 or 16 mg/mL. In particular embodiments, the DNAzym concentration is about 16 mg/mL. In other embodiments, the DNAzym concentration is 8 mg/mL and in a further embodiment, the DNAzym concentration is about 4 mg/mL.

The specific composition and the ratio of the two different poloxamers can influence the gel-forming behaviour of the composition, in addition to the concentration of the DNAzym.

As such, in one embodiment, the composition comprises a total of up to 50% w/w of a combination of light and heavy molecular weight poloxamer. In particular the composition comprises a total of up to 50% w/w of a mixture of poloxamer 407 and poloxamer 188. In some embodiments the total content of poloxamer is in the range of 10% to 50% w/w.

If not specified otherwise, percentage values refer to weight percent, i.e. w/w.

In a particular embodiment the total content of high molecular weight poloxamer and low molecular weight poloxamer is in the range from 20 to 35% w/w. In specific embodiments, the poloxamers are poloxamer 407 and poloxamer 188 and the total content of poloxamer 188 and poloxamer 407 is in the range from 20 to 35% w/w.

The weight ratio of low molecular weight poloxamer and high molecular weight poloxamer can be in any suitable range. In some embodiments, the ratio of low molecular weight poloxamer is in the range of 1:5 to 1:20.

In some embodiments, the composition comprises up to 30% w/w of high molecular weight poloxamer, in particular poloxamer 407. In some embodiments, the composition comprises 18 to 30% w/w of high molecular weight poloxamer, in particular poloxamer 407.

In some embodiments of the invention, the composition comprises about 20 to 25% w/w of high molecular weight poloxamer, in preferred embodiments, the composition comprises about 20 to 25% w/w of poloxamer 407.

In specific embodiments, the composition comprises 22.5% w/w of high molecular weight poloxamer, in particular about 22.5% w/w of poloxamer 407.

In general, it is preferred that the amount of low molecular weight poloxamer is less than 20% w/w. In some embodiments, the composition comprises up to 20% of low molecular weight poloxamer, in preferred embodiments up to 20% of poloxamer 188.

In some embodiments of the invention, the composition comprises about 1 to 10% w/w of low-molecular weight poloxamer, in particular about 1 to 10% w/w of poloxamer 188.

In specific embodiments of the invention, the composition comprises about 2 to 5% w/w of low-molecular weight poloxamer, in particular, the composition comprises about 2 to 5% w/w of poloxamer 188.

In some embodiments of the invention, the composition comprises about 2.5% w/w of a low-molecular weight poloxamer, in particular, in some embodiments the composition comprises about 2.5% w/w of poloxamer 188.

The composition may additionally comprise stabilizers, excipients or other pharmaceutically acceptable compounds.

Suitable stabilizers that can be used include trehalose or diglycine.

If the composition comprises a stabilizer, it is preferred that the composition comprises at most 20% w/w of a stabilizer.

As such, in some embodiments of the invention, the composition comprises of up to 20% of a stabilizer. In some embodiments, the composition comprises about 2 to 16% w/w of stabilizer. In particular embodiments, the wherein the composition comprises about 5% w/w of stabilizer.

The composition comprises a pharmaceutically acceptable buffer. Said buffer may be any pharmaceutically acceptable buffer. Pharmaceutically acceptable buffers are based for example on TRIS, HEPES, phosphate, acetate or citrate. Any suitable buffer may be used in the composition.

In preferred embodiments of the invention the buffer is a phosphate based buffer, for example a PBS buffer. The pH of the buffer is preferably a physiologically and pharmaceutically acceptable pH. In some embodiments, the pH of the buffer is between 7 and 8, in particular embodiments the pH is 7.4. In some embodiments the pH is from 7.2 to 7.6. In some embodiments the pH is selected from 7.0, 7.1, 7.2, 7.3, 7.4, 7.5, 7.6, 7.7, 7.8, 7.9 and 8.0.

In some embodiments, the invention relates to a composition comprising:
a) 20 to 25% w/w poloxamer 407;
b) 2 to 5% w/w poloxamer 188;
c) 2 to 16 mg/mL DNAzym;
d) a phosphate buffer at pH 7.4; and
e) optionally up to 10% w/w of a stabilizer.

In a preferred embodiment, the invention relates to a composition comprising:
a) 22.5% w/w poloxamer 407;
b) 5% w/w poloxamer 188;
c) 8 mg/mL hgd40;
d) a phosphate buffer at pH 7.4.

In a particular embodiment of the invention, the composition comprises:
a) 20% w/w poloxamer 407;
b) 2.5% w/w poloxamer 188;
c) 8 mg/mL hgd40;
d) a phosphate buffer at pH 7.4.

In a further embodiment of the invention, the composition comprises:
a) 22.5% w/w poloxamer 407;
b) 2.5% w/w poloxamer 188;
c) 8 mg/mL hgd40;
d) a phosphate buffer at pH 7.4;
e) 5% w/w trehalose.

In yet a further embodiment, the composition comprises:
a) 22.5% w/w poloxamer 407;
b) 2.5% w/w poloxamer 188;
c) 16 mg/mL hgd40;
d) a phosphate buffer at pH 7.4;

An advantage of the claimed compositions is that the composition have a short gelation time. Accordingly, in one embodiment the invention relates to pharmaceutical composition as defined above, wherein the composition has a gelation time of less than 5 minutes. In some embodiments, the composition has a gelation time between 20 seconds and 5 minutes. In preferred embodiments, the composition has a gelation time of one minute or less. In a particular embodiment, the composition has a gelation time of between 30 to 60 seconds.

In a further aspect to the invention, the invention relates to the use of the compound of the invention as a medicament. Accordingly, in one aspect, the invention relates to a composition as defined above for use as a medicament.

The pharmaceutical composition may be used as a medicament. As noted before, the composition is suitable for rectal application, reduces drug leakage and improves the comfort of the patient during the treatment compared to a normal enema with a liquid aqueous composition. A major advantage of the pharmaceutical composition of the invention is that the composition is a liquid at room temperature and a solid or semisolid gel at about 37° C.

The pharmaceutical composition according to the invention, in particular when comprising a DNAzyme that specifically inhibits GATA-3 expression, such as hgd40, or any one of SEQ ID NO 1 to SEQ ID NO 70.

As such, the invention further relates to a composition as defined above for use in the treatment of an intestinal disease, in particular an inflammatory intestinal disease. In one embodiment, the invention relates to a pharmaceutical composition as defined above for use in the treatment of an inflammatory intestinal disease, wherein said disease is colitis.

The invention further relates to the use of a pharmaceutical composition as defined above in the manufacture of a medicament. In some embodiments said medicament is a medicament for an intestinal disease, in particular an inflammatory intestinal disease. In a particular embodiment, the invention relates to the use of a pharmaceutical composition as defined above in the manufacture of a medicament for colitis, in particular colitis ulcerosa.

In a further aspect of the invention, the invention relates to a method of treating an intestinal disease comprising administering a pharmaceutical composition as defined above to a patient in need thereof.

The following examples serve to illustrate the invention, however should not to be understood as restricting the scope of the invention.

### EXAMPLES

### Example 1—Gel Forming Formulations Studies

In a first step, the inventors prepared hydrogel formulations without DNAzyme to identify potential hydrogel compositions to serve as a basis for the DNAzyme hydrogel formulations.

Hydrogel formulations were prepared using Poloxamer 407 and 188 in either PBS buffer with 120 mM NaCl or 140 mM NaCl.

The gelation time and gel-transition temperature (T_{sol-gel}) were measured. All formulations were prepared in Buffer 1 (120 mM NaCl) and Buffer 2 (140 mM NaCl) and the results were similar for each buffer. The results are presented in table 1.

TABLE 1

| Formulation | 1 | 2 | 3 | 4 |
|---|---|---|---|---|
| Poloxamer 407 (% w/w) | 15 | 15 | 15 | 17.5 |
| Poloxamer 188 (% w/w) | 0 | 5 | 10 | 0 |
| room temperature | Liquid | Liquid | Liquid | Liquid |
| 37° C. | Liquid | Liquid | Liquid | Gel |
| T_{sol-gel} (° C.) | >37 | >37 | >37 | 35-37 |
| Formulation | 5 | 6 | 7 | 8 |
| Poloxamer 407 (% w/w) | 17.5 | 17.5 | 20 | 20 |
| Poloxamer 188 (% w/w) | 2.5 | 5 | 0 | 2.5 |
| room temperature | Liquid | Liquid | Liquid | Liquid |
| 37° C. | Liquid | Liquid | Gel | Liquid |
| T_{sol-gel} (° C.) | >37 | >37 | 26-28 | >37 |
| Formulation | 9 | 10 | 11 | 12 |
| Poloxamer 407 (% w/w) | 20 | 25 | 25 | 25 |
| Poloxamer 188 (% w/w) | 5 | 0 | 5 | 7.5 |
| room temperature | Liquid | Liquid | Liquid | Liquid |
| 37° C. | Liquid | Gel | Gel | Gel |
| T_{sol-gel} (° C.) | >50 | 25-26 | 30 | 33-34 |

| Formulation | 13 | 14 |
|---|---|---|
| Poloxamer 407 (% w/w) | 25 | 25 |
| Poloxamer 188 (% w/w) | 10 | 15 |
| room temperature | Liquid | Liquid |
| 37° C. | Gel | Gel |
| T_{sol-gel} (° C.) | 34-36 | 35-37 |

Formulations 4, 13 and 14 were considered to show the desired behaviour, i.e. a liquid at room temperature and gel-forming at 37° C. with a suitable T_{sol-gel}. As such, these formulations were selected for further analysis.

### Example 2: Gel-Formulations Comprising hgd40

For initial tests formulation 13 was used for initial analysis of hdg40 formulation. 4, 8, 16 and 32 mg of hgd40 were prepared in 1 mL PBS buffer (120 mM NaCl), with 25% w/w of Poloxamer 407 and 10% w/w of Poloxamer 188.

It was found that hgd40 was not soluble in the formulation, even in 4 mg/ml concentration and all formulations were turbid at room temperature. Therefore formulation 4 was further investigated.

The new formulation comprised 17.5% w/w Poloxamer 407, 0% Poloxamer 188 and 4 mg hgd40 in PBS buffer with 120 mM NaCl. The DNAzyme was soluble in the formulation. However, in contrast to the formulation without hgd40, the formulation did not form a hydrogel.

A formulation comprising 20% w/w Poloxamer 407 and 2.5% w/w Poloxamer 188, was able to form a gel comprising 4 mg/mL hgd40.

An overview on the formulations is found in table 2.

TABLE 2

| Formulation | 15 | 16 | 17 |
|---|---|---|---|
| Poloxamer 407 (% w/w) | 25 | 17.5 | 20 |
| Poloxamer 188 (% w/w) | 10 | 0 | 2.5 |
| hgd40 (mg/mL) | 32, 16, 8 or 4 | 4 | 4 |
| | (insoluble) | | |
| room temperature | Liquid | Liquid | Liquid |
| 37° C. | nd | liquid | Gel |
| T_{sol-gel} (° C.) | nd | no transition | 36.6 |

### Example 3: hgd40 Hydrogel Release Studies

In order to analyse the release of hgd40 from the hydrogel composition 17 above, 100 mL of Buffer (PBS, 120 mM NaCl) were stirred at 37° C. in a glass vial. 1 mL of the hydrogel formulation was loaded in a dialysis tube (MWCO 300 kD, Float-A-Lyzer G2, sprectrumlabs) and placed in the glass vial. A composition comprising 4 mg hgd40 in the buffer was used as a control.

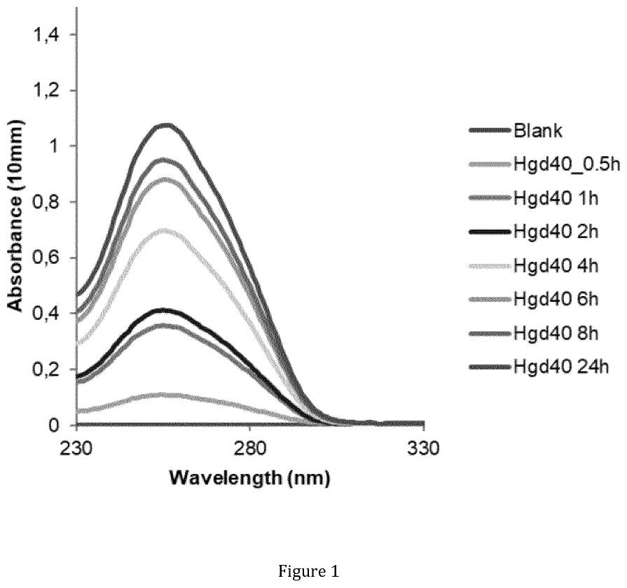

After 30 min, 1 h, 2 h, 4 h, 6 h, 8 h and 24 h a sample was taken from the buffers and analysed by UV spectroscopy between 230-330 nm. The resulting spectra for the hgd40 samples are found in FIG. 1.

The concentration of hgd40 in the buffer was determined using the absorbance at 260 nm and applying the Lambert-Beer law. The concentrations released from the compositions are listed in Table 3.

TABLE 3

| | Control | Hydrogel |
|---|---|---|
| Time (h) | Conc. hgd40 (μg/mL) | Conc. hgd40 (μg/mL) |
| 0.5 | 1.00 | 3.70 |
| 1 | 2.18 | 12.76 |
| 2 | 5.77 | 14.69 |
| 4 | 15.39 | 24.82 |
| 6 | 23.49 | 31.48 |
| 8 | 29.81 | 33.99 |
| 24 | 46.09 | 38.40 |

It was found that hgd40 was released from both control and formulation F34 at a similar release rate. As such, the hydrogel formulation allows for a similar release as an aqueous composition.

The hgd40 in buffer before release and the released hgd40 were analysed using AEX chromatography to analyse if the hydrogel formulation would result in denaturation of the DNAzyme.

The AEX chromatography conditions were as follows:
Column DNAPac™ PA200, 8 μm, 4×250 mm
Buffers:
A: 25 mM TRIS/HCl (pH 8), 1 mM EDTA, 30% ACN
B {25 mM TRIS/HCl (pH 8), 1 mM EDTA, 30% ACN, 500 mM LiClO4
Column Temperature 40° C.
Flow Rate 1 mL/min
Detection: 260/280 nm

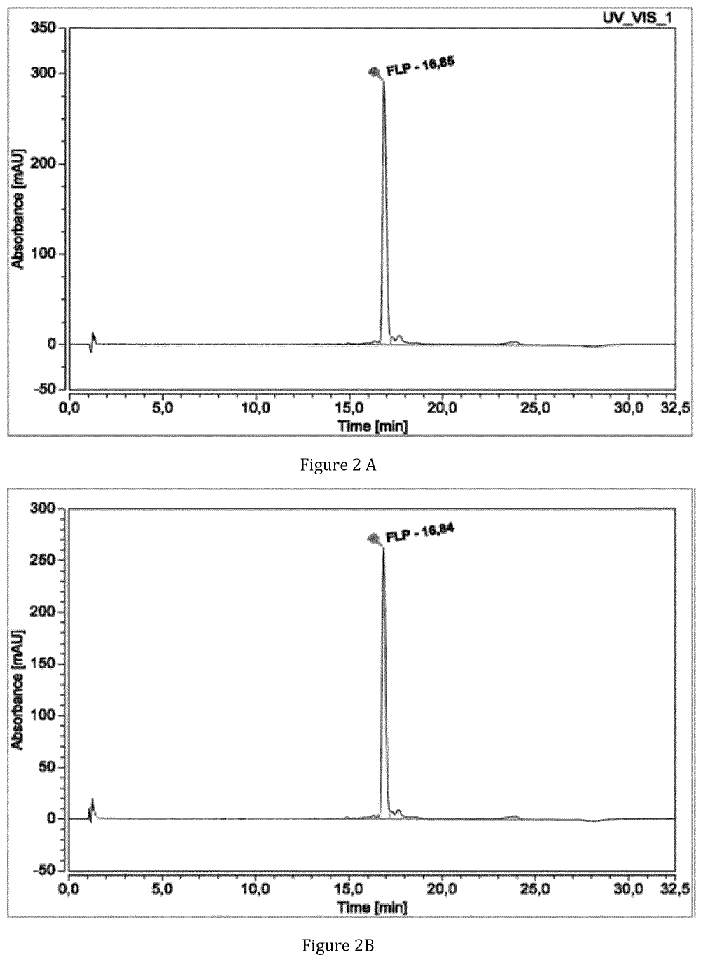

As seen in FIGS. 2A and 2 B, the chromatography profile of HPLC after release from the hydrogel is similar to the profile of the stock solution, indicating that no denaturation occurred and the hgd40 DNAzyme is stable in the hydrogel formulation.

### Example 4: Increasing DNAzym Concentration

As a next step, the inventors increased the hgd40 concentration in the composition to 8 mg/mL. The formulations were prepared in 100 mM sodium phosphate buffer at different pH. The concentration of hgd40 in the formulation is 8 mg/mL.

The different pH buffer solutions were obtained by mixing different ratios of 200 mM Na₂HPO₄ and 200 mM NaH₂PO₄ stock solutions and dilution with water to obtain a 100 mM buffer.

An overview on the tested formulations is found in table 4.

TABLE 4

| Formulation | 18 | 19 | 20 | 21 |
|---|---|---|---|---|
| Poloxamer 407 (% w/w) | 20 | 20 | 20 | 20 |
| Poloxamer 188 (% w/w) | 2.5 | 2.5 | 2.5 | 2.5 |
| buffer pH | 6.2 | 6.8 | 7.4 | 8.0 |
| room temperature | Liquid | Liquid | Liquid | Liquid |
| 37° C. | Liquid | Liquid | Liquid | Liquid |

It was found that the compositions with 8 mg/mL hgd40 did not form a gel at 37° C.

As such, the inventors tried to optimize the formulations comprising 8 mg/ml hdg40.

An overview on the new formulations can be found in table 5.

As a buffer 100 mM Sodium phosphate at pH 7.4 was used.

TABLE 5

| Formulation | 21 | 22 | 23 | 24 | 25 |
|---|---|---|---|---|---|
| Poloxamer 407 (% w/w) | 20 | 22.5 | 25 | 22.5 | 22.5 |
| Poloxamer 188 (% w/w) | 2.5 | 2.5 | 2.5 | 5 | 10 |
| hgd40 | dis- | dis- | dis- | dis- | not dis- |
| | solved | solved | solved | solved | solved |
| room temperature | Liquid | Liquid | Liquid | Liquid | Liquid |
| 37° C. | Liquid | Gel | Gel | Gel | gel |
| T_{sol-gel} (° C.) | nd | 31 | <30 | 36 | 41 |

Composition 25 was further tested with 16 mg/mL hgd40, which caused hgd40 to precipitate, which resulted in a turbid solution. The solution clears when warmed to 37° C., and returns to turbid upon cooling down. Further, T_{sol-gel} increased to 41° C. with 16 mg/mL hgd40.

Further formulations were prepared to provide compositions which allow for a higher hdg40 concentration. In order to reduce influence of salt and other compounds, hdg40 was purified by desalted using a SEC column, and subsequently lyophilized and dissolved in 100 mM sodium phosphate buffer pH 7.4.

Table 6 shows an overview on the compositions prepared.

TABLE 6

| Formulation | 26 | 27 | 28 |
|---|---|---|---|
| Poloxamer 407 (% w/w) | 22.5 | 22.5 | 22.5 |
| Poloxamer 188 (% w/w) | 2.5 | 2.5 | 2.5 |
| hgd40 conc. (mg/mL) | 2 | 6 | 18 |
| hgd40 | dissolved | dissolved | dissolved |
| room temperature | Liquid | Liquid | Liquid |
| 37° C. | Gel | Gel | Liquid |
| T_{sol-gel} (° C.) | 28 | 30 | nd |

| Formulation | 29 | 30 | 31 | 32 |
|---|---|---|---|---|
| Poloxamer 407 (% w/w) | 22.5 | 22.5 | 25 | 22.5 |
| Poloxamer 188 (% w/w) | 5 | 5 | 2.5 | 2.5 |
| hgd40 conc. (mg/mL) | 2 | 6 | 18 | 16 |
| hgd40 | dissolved | dissolved | not | dissolved |
| | | | dissolved | |
| room temperature | Liquid | Liquid | Liquid | Liquid |
| 37° C. | gel | Gel | Gel | Gel |
| T_{sol-gel} (° C.) | 33 | 34 | 31 | >40 |

| Formulation | 33 | 34 | 35 |
|---|---|---|---|
| Poloxamer 407 (% w/w) | 23 | 23 | 23 |
| Poloxamer 188 (% w/w) | 5 | 5 | 2.5 |
| hgd40 conc. (mg/mL) | 2 | 6 | 16 |
| hgd40 | dissolved | dissolved | dissolved |
| room temperature | Liquid | Liquid | Liquid |
| 37° C. | gel | Gel | Gel |
| T_{sol-gel} (° C.) | 31 | 32 | >40 |

### Example 5: Effect of Stabilizers Trebalose and Digylcin

The formulations were prepared in 100 mM sodium phosphate buffer at pH 7.4 at two different poloxamer 407 concentrations. For each poloxamer 407 concentration, formulations were prepared with 2 different trehalose concentrations (5 and 15%).

TABLE 7

| Formulation | 26 | 27 | 28 | 29 |
|---|---|---|---|---|
| Poloxamer 407 (% w/w) | 20 | 20 | 22.5 | 22.5 |
| Poloxamer 188 (% w/w) | 2.5 | 2.5 | 2.5 | 5 |
| Trehalose (% w/w) | 5 | 15 | 5 | 15 |
| room temperature | Liquid | Liquid | Liquid | Liquid |
| 37° C. | Liquid | Liquid | Gel | Liquid |
| T_{sol-gel} (° C.) | nd | nd | 34.8 | >50 |

At low trehalose concentration (5%) and increasing poloxamer 407 amount, a hydrogel could be formed at 37° C. (Formulation 29). This formulation has T_{sol-gel} around 35° C. At high trehalose concentration, independent on the amount of poloxamer 407, the formulations were liquid at 37° C.

A formulation with diglycide was prepared as comparison.

TABLE 8

| Formulation | 30 |
|---|---|
| Poloxamer 407 (% w/w) | 20 |
| Poloxamer 188 (% w/w) | 2.5 |
| diglycide (% w/w) | 5 |
| room temperature | Liquid |
| 37° C. | Liquid |
| T_{sol-gel} (° C.) | >50 |

The composition with diglycide as a stabilizer showed an increased T_{sol-gel} compared to a composition with trehalose or without stabilizer.

### Example 6: Gelation Times

The formulations were prepared in 100 mM sodium phosphate buffer at pH 7.4. The poloxamers F127, F68 and Hgd40 were weighed in a glass vial (see table 1), to the mixture was added 1 g of 100 mM Na2HPO4/NaH2PO4 buffer pH 7.4. The dissolution was performed at 4° C. until a clear solution was obtained and took place under continuous and slow stirring for 24 to 48 h.

Sol-gel transition temperature (Tsol-gel):

The sol-gel transition temperature was determined by the “magnetic stirrer method”. The poloxamer formulation was loaded into a transparent vial containing a magnetic bar in a thermostatic water bath at room temperature, and a digital thermo-sensor was immersed into the formulation. The sample was gradually heated (2° C./min) while being continuously stirred (100 rpm/min) on a magnetic stirrer (IKA® RCT basic). When the magnetic bar stops moving due to gelation, the temperature displayed on the thermo-sensor was identified as the gelation temperature.

### Gelation Time:

The poloxamer formulation was loaded into a transparent vial containing a magnetic bar in a thermostatic water bath at 37° C., and a digital thermo-sensor was immersed into the formulation. The sample was warmed up to 37° C. under stirring (100 rpm/min) on a magnetic stirrer (IKA® RCT basic). When the magnetic bar stops moving due to gelation, the time recorded from the loading until gelation was identified as the gelation time.

The results are shown in table 9.

TABLE 9

| Formulation | 31 | 32 | 33 |
|---|---|---|---|
| Poloxamer 407 (% w/w) | 23 | 23 | 23 |
| Poloxamer 188 (% w/w) | 5 | 5 | 5 |
| hgd40 conc. (mg/mL) | 2 | 6 | 16 |
| Tsol-gel (° C.) | 30.6 | 31.0 | 32.5 |
| Gelation time at 37° C. (s) | 35.0 | 40.0 | 51.0 |

1. A pharmaceutical composition comprising:
a) a high molecular weight poloxamer;
b) a low molecular weight poloxamer;
c) a DNAzyme;
d) a pharmaceutically acceptable buffer;
wherein the composition is liquid at room temperature and solid or semisolid at a temperature of 36° C. or higher.

2. The pharmaceutical composition according to claim 1, wherein the content of the DNAzyme is in the range from 4 mg/mL to 20 mg/mL.

3. The pharmaceutical composition according claim 1, wherein the high molecular weight poloxamer is poloxamer 407 and/or the low molecular weight poloxamer is poloxamer 188.

4. The pharmaceutical composition according to claim 1, wherein the high molecular weight poloxamer is poloxamer 407, and wherein the composition comprises about 18 to 30% w/w of poloxamer 407.

5. The pharmaceutical composition according to claim 1, wherein the low molecular weight poloxamer is poloxamer 188, and wherein the composition comprises about 1 to 10% w/w of poloxamer 188.

6. The pharmaceutical composition according to claim 1, wherein the high molecular weight poloxamer is poloxamer 407, wherein the low molecular weight poloxamer is poloxamer 188, and wherein the weight ratio of poloxamer 188 to poloxamer 407 is in the range from 1:5 to 1:20.

7. The pharmaceutical composition according to claim 1, additionally comprising a stabilizer, optionally wherein the stabilizer is trehalose or digylcin.

8. The pharmaceutical composition according to claim 1, wherein the DNAzyme is selected from Seq ID No 1 to Seq ID No 70, preferably wherein the DNAzyme is hgd40 (Seq ID No. 40).

9. The pharmaceutical composition according to claim 1, wherein the pharmaceutically acceptable buffer is a phosphate buffer with a pH between 7 and 8.

10. The pharmaceutical composition according to claim 1, wherein the composition comprises:
a) 20 to 25% w/w poloxamer 407;
b) 2 to 5% w/w poloxamer 188;
c) 2 to 16 mg/mL DNAzyme;
d) a phosphate buffer with a pH between 7 and 8; and
e) up to 10% w/w of a stabilizer.

11. The pharmaceutical composition according to claim 1, wherein the composition comprises:
a) 20 to 25% w/w poloxamer 407;
b) 2 to 5% w/w poloxamer 188;
c) 2 to 16 mg/mL DNAzyme;
d) a phosphate buffer with a pH between 7 and 8.

12. The pharmaceutical composition according to claim 1, wherein the composition comprises:
a) 22.5% w/w poloxamer 407;
b) 2.5% w/w poloxamer 188;
c) 16 mg/mL hgd40;
d) a phosphate buffer at pH 7.4.

13. The pharmaceutical composition according to claim 1, wherein the composition has a gelation time of less than 5 minutes.

14. The pharmaceutical composition according to claim 1, wherein the composition has a gelation time between 20 seconds and 5 minutes.

15. The pharmaceutical composition according to claim 1, wherein the composition has a gelation time of one minute or less.

16. The pharmaceutical composition according to claim 1, wherein the composition has a gelation time of between 30 to 60 seconds.

17. A method of treating a disease, the method comprising administering a pharmaceutical composition according to claim 1 to a patient in need thereof.

18. The method according to claim 17, wherein the disease is an intestinal disease.

19. The method according to claim 18, wherein the intestinal disease is an inflammatory intestinal disease.

20. The method according to claim 17, wherein the composition is administered rectally.
